(19) United States
(12) Patent Application Publication
(10) Pub. No.: US 2005/0084893 A1
(43) Pub. Date: Apr. 21, 2005

(54) Automated bioaerosol analysis platform

(75) Inventors: Robert Alan Herman, Baltimore, MD (US); Jared Thomas Ackers, Baltimore, MD (US); Douglas Jason Green, Baldwin, MD (US)

Correspondence Address: FOLEY AND LARDNER;SUITE 500, 3000 K STREET NW, WASHINGTON, DC, 20007, US

(73) Assignee: SMITHS DETECTION INC.

(21) Appl. No.: 10/962,480

(22) Filed: Oct. 13, 2004

Related U.S. Application Data
(60) Provisional application No. 60/511,426, filed on Oct. 16, 2003.

(51) Int. Cl. C12Q 1/68; C12M 1/34

(52) U.S. Cl. 435/6; 435/287.1

(57) ABSTRACT

A system for generating a liquid sample includes a chamber adapted to hold a fluid, an air filter configured to be received in the chamber, a mechanism for releasing at least a portion of a particulate disposed on the filter into the fluid located in the chamber, and a structure for removing at least a portion of the particulate containing fluid from the chamber.

### CROSS-REFERENCE TO RELATED PATENT APPLICATIONS

This application claims priority to and the benefit of U.S. Provisional Application No. 60/511,426, filed Oct. 16, 2003, and incorporated by reference herein.

### BACKGROUND OF THE INVENTION

The present invention relates generally to detection and identification of bioaerosols and, more particularly, to a system for washing a filter to release biological particles that are entrained in the filter.

Infectious biological particles such as bacteria and viruses can be transferred from one organism (e.g., a human or animal) to another via an airborne route. For example, biological particles can inadvertently become aerosolized into bioaerosols when a person speaks, coughs, or sneezes or during certain medical and dental procedures that generate particle-containing droplets. Biological particles can also exist, for example, in vaporized water from cooling towers, water faucets, and humidifiers; in agricultural dust; and in other airborne organic materials.

In addition to bioaerosols that are produced inadvertently from common sources, bioaerosols can be generated intentionally. For example, individuals bent on harming others and disrupting society have demonstrated that hazardous biological particles, such as anthrax in micron-sized particles, can be spread in envelopes delivered through the postal system. Such particles can become airborne during processing in postal facilities or when a contaminated envelope is opened. For example, in October 2001, anthrax was discovered in mail processed by the United States Postal Service in Washington, D.C., resulting in serious illness to postal employees and at least two deaths. In October 2001, anthrax was also discovered in the mail room and office buildings of the Unites States Capitol resulting in closure and quarantine of the buildings. Other methods of intentionally distributing and aerosolizing hazardous biological particles include, for example, dispersing particles through ventilation systems or by explosive release.

In order to protect humans and animals from illness caused by inhalation of hazardous bioaerosols, systems to monitor, detect, and identify bioaerosols exist. One commonly used method for monitoring, detecting,and identifying hazardous bioaerosols employs dry filter devices (e.g., air filters) that are manually collected and analyzed using laboratory procedures. The laboratory procedures involve washing the filters using physical agitation, then performing standard laboratory processes (such as centrifuge) to prepare the sample for analysis. Manually collecting and analyzing the filters, however, presents a logistical burden. Moreover, because the collection and analysis systems involve separate components, conventional methods are not well-suited for use in non-laboratory environments. As a result, such systems are not adapted for use by facility security professionals, military forces, and first responders, such as fire fighters, police, emergency medical personnel, and HAZMAT teams, to determine whether a life threatening biohazard is present at locations on-site and in the field.

Although automated collection and identification systems exist, such systems typically employ wet-walled aerosol collectors or similar devices, which require greater amounts of liquid consumables than a dry filter device. For example, wet-walled aerosol collectors and similar devices typically require significant amounts of liquid reagents during a collection cycle in a high temperature environment because the collection fluids evaporate as a result of the high temperature and have to be replenished. Additionally, in low temperature environments, wet-walled aerosol collectors and similar devices require the use of means to prevent the collection fluid or sample air flow from freezing during collection. For example, the collection fluid may be heated. Heating the collection fluid (or employing other means to prevent the collection fluid from freezing), however, imposes additional power requirements on the system.

Another disadvantage of wet-walled aerosol collectors (or similar devices) is that such devices typically have a low retention factor because collected particles re-aerosolize out of the fluid after being collected. As a result, the amount of sample that can be collected over time is reduced.

### SUMMARY OF THE INVENTION

According to an embodiment of the present invention, a system for generating a liquid sample is provided. The system includes a chamber adapted to hold a fluid, an air filter configured to be received in the chamber, a mechanism for releasing at least a portion of a particulate disposed on the filter into the fluid located in the chamber, and a structure for removing at least a portion of the particulate containing fluid from the chamber.

According to another embodiment, a cartridge for processing a liquid sample is provided. The cartridge includes a chamber adapted to hold a fluid, a filter received in the chamber, an inlet for percolating air through the filter to thereby release a particulate disposed on the filter into the fluid, and an outlet for transferring the particulate containing fluid from the chamber.

According to yet another embodiment, a method for generating a liquid sample is provided. The method includes collecting a particulate on a filter, submerging the filter in a fluid, percolating a gas through the filter so that the particulate is washed from the filter into the fluid, and transferring at least a portion of the particulate containing fluid into a reservoir to thereby generate the liquid sample.

It is to be understood that both the foregoing general description and the following detailed description are exemplary and explanatory only, and are not restrictive of the invention as claimed.

### BRIEF DESCRIPTION OF THE DRAWINGS

The accompanying drawings, which are incorporated in and constitute a part of this specification, illustrate exemplary embodiments of the invention and, together with the description, serve to explain principles of the invention.

FIG. 1 is a perspective view of an embodiment of a filter washing assembly according to the present invention.

FIG. 2 is a cross sectional perspective view of the filter washing assembly of FIG. 1 taken along the line 2-2.

FIG. 3A is a perspective view of a lid of the filter washing assembly of FIG. 1.

FIG. 3B is a perspective view of a base of the filter washing assembly of FIG. 1.

FIG. 4A is a cross sectional side elevational view of the lid of FIG. 3A taken along the line 4A-4A.

FIG. 4B is a cross sectional side elevational view of the base of FIG. 3B taken along the line 4B-4B.

FIG. 4C is a cross sectional side elevational view showing the lid of FIG. 3A and the base of FIG. 3B connected together and including fluid and a filter.

FIG. 5 is a perspective view of another embodiment of a filter according to the present invention showing a particulate and a control agent entrained in the filter.

FIG. 6 is a perspective view of another embodiment of a filter washing assembly according to the present invention.

FIG. 7 is a perspective view of another embodiment of a filter washing assembly according to the present invention.

FIG. 8 is a perspective view of another embodiment of a filter washing assembly according to the present invention.

FIG. 9 is a schematic block diagram showing an embodiment of a filter washing assembly and mechanism according to the present invention.

FIG. 10 is a schematic block diagram showing another embodiment of a filter washing assembly and mechanism according to the present invention.

FIG. 11 is schematic block diagram showing another embodiment of a filter washing assembly and mechanism according to the present invention.

FIG. 12 is a flow chart showing a method of washing a filter according to an embodiment of the present invention.

FIG. 13 is a flow chart showing another method of washing a filter according to an embodiment of the present invention.

### DETAILED DESCRIPTION

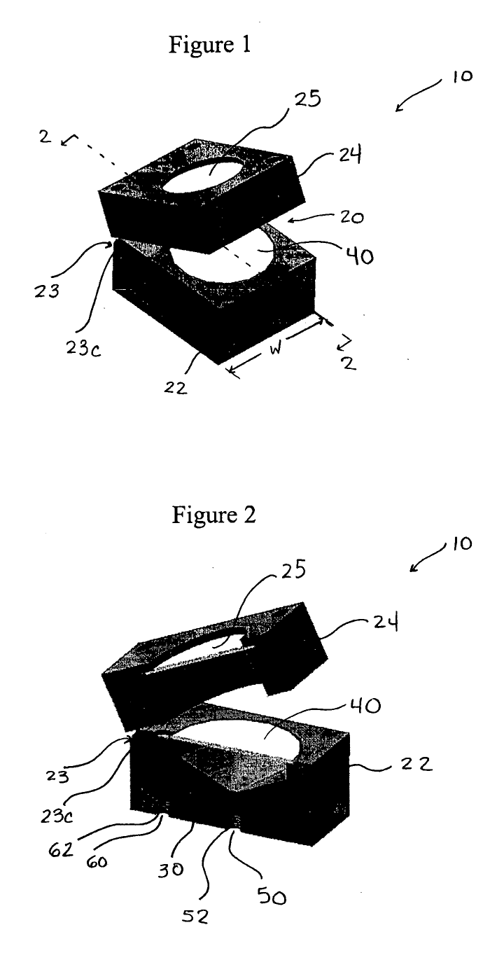

FIGS. 1-4C show an embodiment of a filter washing assembly 10 according to the present invention. The filter washing assembly 10 includes a housing 20, a chamber 30, a filter 40, an inlet 50, and an outlet 60.

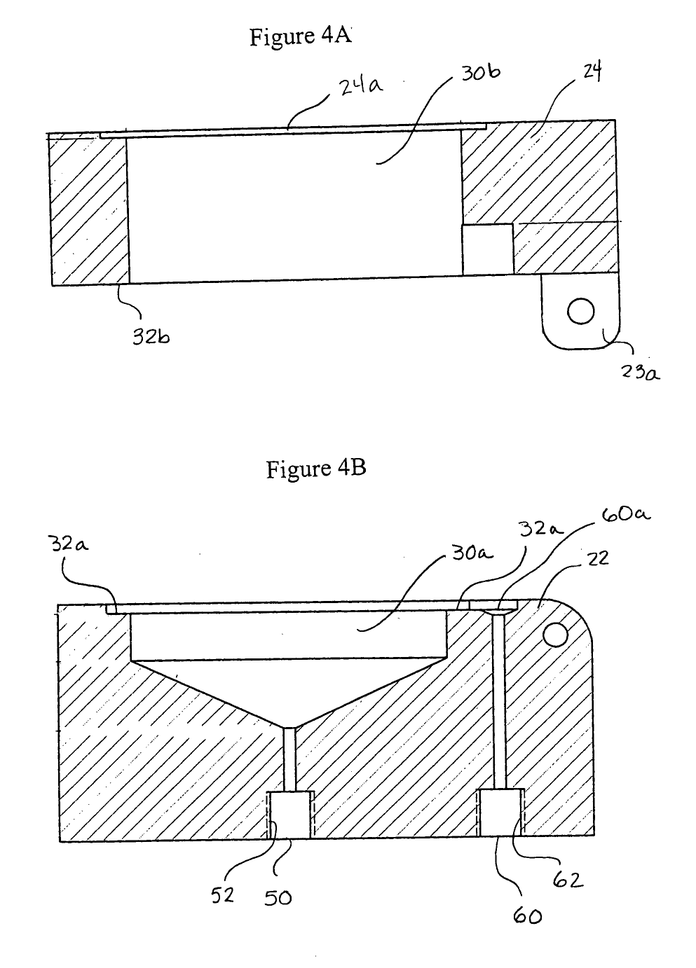

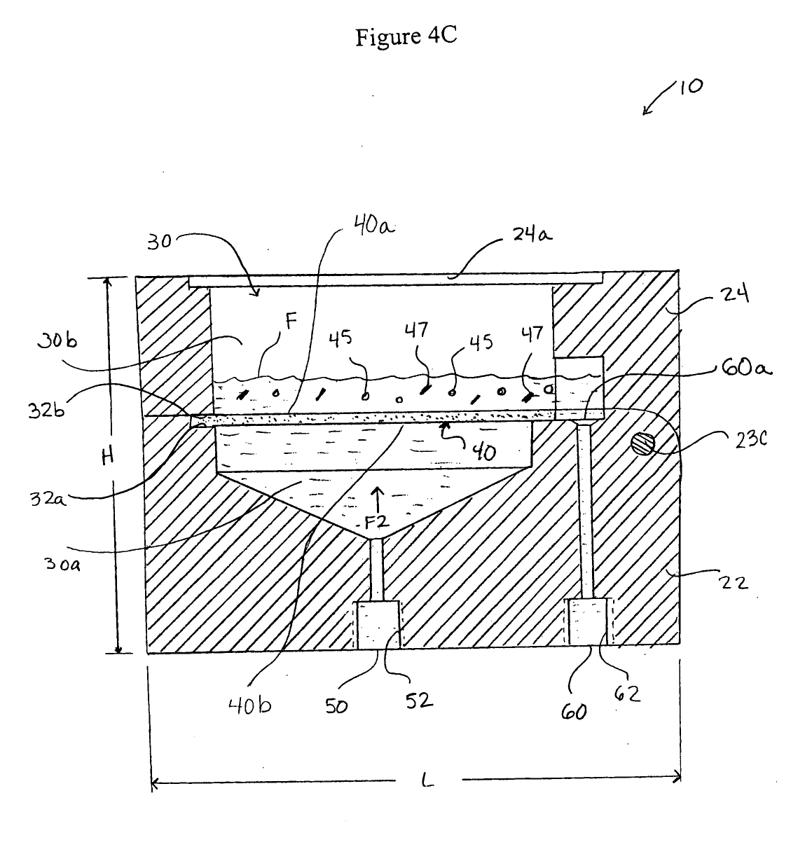

The housing 20 may include a base 22 and a lid 24. The lid 24 is connected to the base 22 so that the lid 24 may be moved from a closed position (shown in FIG. 4C) to an open position (shown in FIG. 1) to provide access to the filter 40. For example, the lid 24 may be connected to the base 22 by a hinge mechanism 23. The hinge mechanism 23 may include a male element 23a disposed on the lid 24 and a female element 23b disposed on the base 22. As shown in FIGS. 1 and 4C, the male and female elements 23a and 23b may be connected by a rod 23c that enables the lid 24 to pivot between the open and closed positions. Alternatively, the base 22 and the lid 24 may be configured to engage by a sliding, snap, or screw-type connection or may be integral.

The housing 20 may be made of metal or plastic. In an exemplary embodiment, the housing 20 is made of TEFLON®. The housing 20 may be sized so that the filter washing assembly 10 can be integrated into a fully automated microfluidic system such as the Autonomous Pathogen Detection System (APDS) developed by Lawrence Livermore National Laboratories. The dimensions of the housing 20 may also be scaled depending on the size of the filter 40, which is dependent on system performance requirements such as sensitivity. According to one embodiment, a height H of the housing 20 may be approximately 2 inches, a width W of the housing 20 may be approximately 2.25 inches, and a length L of the housing 20 may be approximately 2.75 inches.

The chamber 30 is formed in the housing 20 and is adapted to hold a fluid F. For example, the base 22 of the housing 20 may include a cavity 30a, and the lid 24 of the housing 20 may include a cavity 30b. As shown in FIG. 4C, when the lid 24 is in the closed position, the cavities 30a and 30b align to create the chamber 30. In an exemplary embodiment, the chamber 30 has a cylindrical shape with a conical bottom (as illustrated in FIG. 4C) to reduce the volume of the fluid F required for washing while increasing the surface area of the filter 40 penetrated by the percolation gas.

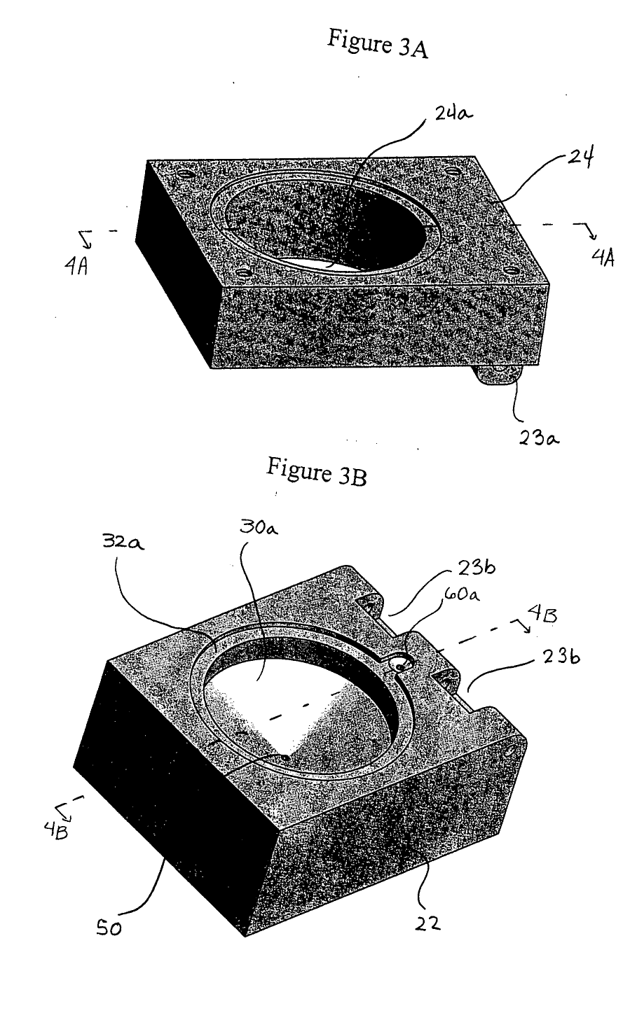

The chamber 30 is configured to receive the filter 40. For example, the chamber 30 may include a ledge 32a (shown in FIG. 3B) disposed on the base 22 and a corresponding ledge 32b (shown in FIG. 4A) disposed on the lid 24. The ledges 32a and 32b support the filter 40 so that the filter 40 extends across the chamber 30 and is secured in the chamber 30 as shown in FIGS. 1 and 2. The filter 40 may be installed in the chamber 30 when the chamber is empty (i.e., when the chamber 30 does not contain fluid). For example, the filter 40 may be installed in the chamber 30 by opening the lid 24 of the housing 20, placing the filter 40 on the ledge 32a, and closing the lid 24 so that the filter is maintained on the ledge 32a by the ledge 32b.

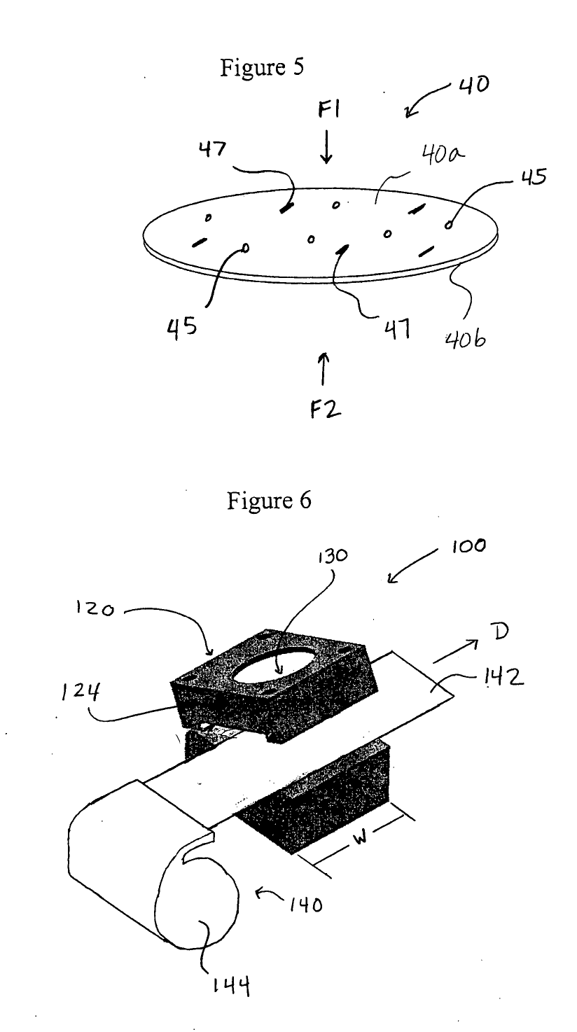

In an exemplary embodiment, the filter washing assembly 10 is configured so that when the filter 40 is installed in the chamber 30, the direction of gas percolation (direction F2 in FIG. 5) is opposite to the direction of sample collection (direction F1 in FIG. 5). For example, as illustrated in FIG. 5, during sample collection, the filter 40 may be disposed so that a first side 40a of the filter 40 receives a flow of air flowing in the direction F1 so that particulate is captured on the first side 40a of the filter 40. When the filter 40 is installed in the chamber 30, the filter 40 may be disposed so that a second side 40b of the filter 40 faces toward the direction F2 of gas percolation. During washing, the gas enters the filter 40 from the second side 40b and exits the filter 40 from the first side 40a thereby dislodging particles trapped on the first side 40a of the filter 40. Additionally, to prevent the particles from becoming re-aerosolized and exiting the filter washing assembly 10, the lid 24 of the housing 20 may optionally include a second filter 25 disposed across an aperture 24a.

The filter 40 is configured to capture airborne particulate and to be received in the chamber 30 so that the particulate captured on the filter 40 may be washed. For example, as shown in FIGS. 2 and 5, the filter 40 may be a dry filter device (e.g., an air filter) having a circular shape with an outer diameter that is approximately equal to an outer diameter of the ledge 32a of the chamber 30. The filter 40 may be made of any material capable of capturing micron-sized particulate, including biological particles such as cells, spores, viruses, toxins, and microorganisms. For example, the filter 40 may be a polyester felt filter, a porous membrane filter, or a glass fiber filter. In an exemplary embodiment, the filter 40 is a polyester felt filter with a 1.0 micron rating. Particulate collection may be performed, for example, by exposing the filter 40 to a flow of air prior to installing the filter 40 in the chamber 30. For example, the filter 40 may be an HVAC filter removably disposed in an air handling system.

The filter 40 may optionally include a control agent 47 (shown in FIG. 5). The control agent 47 is embedded in the filter 40 to verify proper operation of the filter washing assembly 10 and method. For example, the control agent 47 may include a fluorescent dye or polystyrene beads with bound deoxyribonucleic acid segments. When the filter 40 is washed to release the particulate 45, at least a portion of the control agent 47 will also be washed from the filter 40. Thus, a liquid sample generated by washing the filter 40 will include both the particulate 45 and the control agent 47. When the liquid sample is analyzed to determine whether a biological particulate is present and to identify the biological particulate, the presence of the control agent 47 in the liquid sample verifies proper washing of the filter 40. In other words, the presence of the control agent 47 confirms that the filter 40 was washed with sufficient force and for a sufficient length of time to release the particulate 45 trapped in the filter 40. Conversely, an absence of the control agent 47 in the liquid sample indicates that the particulate 45 may not have been washed from the filter 40. Thus, inclusion of the control agent 47 in the filter 40 guards against a false negative reading (i.e., falsely indicating the absence of a biological particle) when the liquid sample is analyzed.

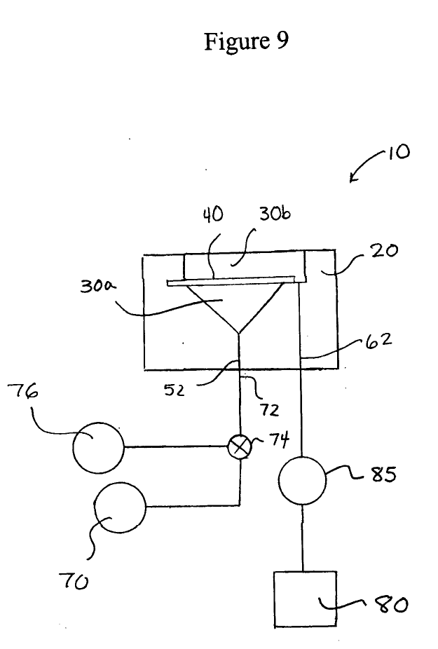

The inlet 50 of the filter washing assembly 10 provides a pathway in the housing 20 from an exterior of the housing 20 to the chamber 30. The inlet 50 functions as a fluid inlet to enable the fluid F (e.g., sterilized water) to be added to the chamber 30 (e.g., by a fluid pump). The inlet 50 additionally enables the housing 20 to be connected to a mechanism 70 (shown in FIG. 9). The mechanism 70 functions to release (or dislodge) at least a portion of the particulate 45 disposed on the filter 40 into the fluid located above the filter 40. The mechanism 70 may be, for example, an air pump that enables a flow of gas (e.g., air) to be supplied to the chamber 30. For example, after the chamber 30 has been filled with fluid, the flow of gas can be delivered into the chamber 30 through the inlet 50 in the direction F2. The gas percolates through the fluid F and the filter 40. As the gas penetrates the filter 40, the gas agitates the filter 40 thereby washing particulate 45 disposed on the filter 40 into the fluid located above the filter 40 as shown in FIG. 4C. If the filter 40 includes the control agent 47, the percolating gas also dislodges the control agent 47 so that the control agent 47 is washed into the fluid. As shown in FIGS. 4C and 5 and discussed above, the flow of gas is in the direction F2, which is opposite to the direction F1 of sample collection, so that the ability of the gas to dislodge (or wash) particulates from the filter 40 is improved.

The inlet 50 includes a fitting 52 configured to couple with a corresponding fitting 72, which may be connected directly or indirectly to the mechanism 70. In this manner, the inlet 50 and the mechanism 70 may be connected together as shown schematically in FIG. 9. The fittings 52 and 72 may be any known coupling mechanism such as a threaded connection. The fitting 72 may be indirectly coupled to the mechanism 70 by a valve 74 (e.g., a two-way valve) so that the inlet 50 can be simultaneously connected to the mechanism 70 and to a fluid supply source 76 such as a fluid pump. In operation, the valve 74 may be actuated to supply fluid from the fluid supply source 76 or gas from the mechanism 70 to the chamber 30. Alternatively, the entire housing 20 may be integrated into a microfluidic manifold thereby eliminating the need for fittings.

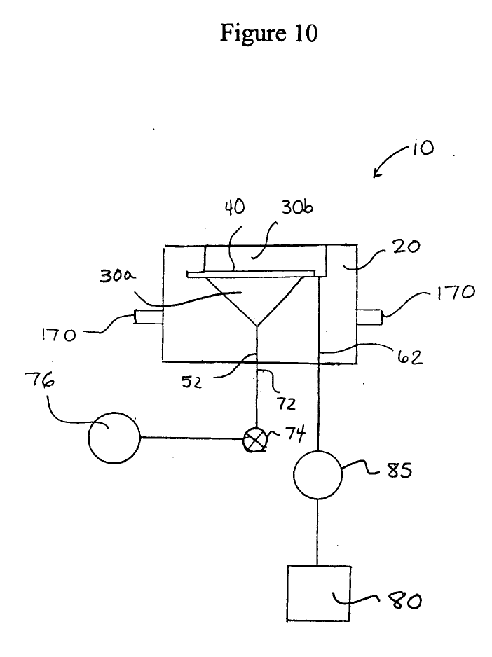

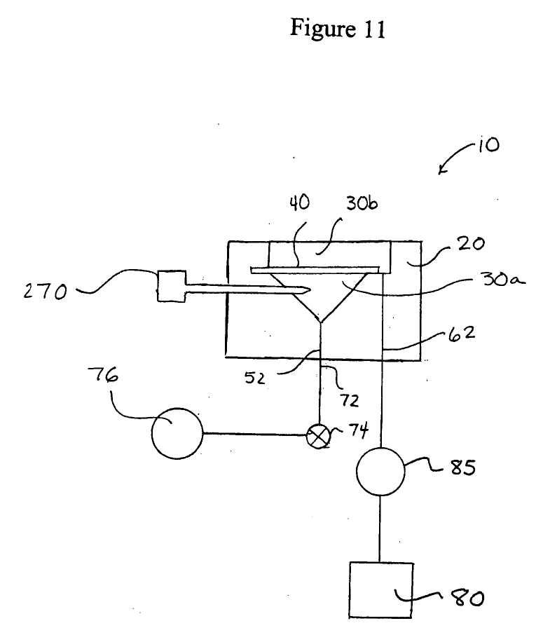

As an alternative to an air pump that supplies a flow of gas to the chamber 30, the mechanism for releasing the particulate may be an agitator adapted to mechanically agitate the filter 40 and/or the filter washing assembly 10. For example, as shown in FIG. 10, the filter washing assembly 10 may be coupled to a mechanical agitator 170. The mechanical agitator 170 agitates the filter 40 to thereby release the particulate 45 from the filter 40. Alternatively, the mechanism for releasing the particulate may be a sonicator that imparts vibrational energy to the fluid. For example, as shown in FIG. 11, an ultrasonic horn 270 may be introduced to the chamber 30 via a channel in the housing 20. Vibrational energy generated by the ultrasonic horn 270 radiates through the fluid and agitates the filter 40. The sonicator may also induce cavitation resulting in the formation of vapor bubbles in the fluid that percolate through the filter 40 to release the particulate 45.

The outlet 60 of the filter washing assembly 10 provides a pathway in the housing 20 from the chamber 30 to an exterior of the housing 20. The outlet 60 enables particulate-containing fluid in the chamber 30 to be transferred out of the chamber 30. For example, after a period of time (e.g., 30 seconds), the particulate laden fluid F above the filter 40 may transferred out of the chamber 30 through the outlet 60 to a reservoir 80. As shown in FIG. 4C, an entrance 60a of the outlet 60 is disposed in the chamber 30 at substantially the same level as the filter 40. Accordingly, when the level of fluid F in the chamber 30 is above the level of the entrance 60a, particulate laden fluid F located above the filter 40 will enter the outlet 60 via the entrance 60a and will be transferred from the chamber 30 through the outlet 60 by gravity. To enhance the transfer of the fluid F from the chamber 30, the filter washing assembly 10 may optionally include a device 85 disposed between the outlet 60 and the reservoir 80. The device 85 may be configured to introduce a suction force at the outlet 60 to aspirate or pump the particulate laden fluid F from the chamber 30 to the reservoir 80. For example, the device 85 may be an aspirator, a peristaltic pump, or a solenoid metering pump.

The outlet 60 includes a fitting 62 configured to couple with a corresponding fitting 82 as shown in FIG. 9. The fitting 82 may be connected to the reservoir 80 or to the transfer device 85 if the filter washing assembly 10 includes a transfer device 85. The fittings 62 and 82 may be any known coupling mechanism such as a threaded connection. Alternatively, the entire housing 20 may be integrated into a microfluidic manifold thereby eliminating the need for fittings. The reservoir 80 may be any container or chamber capable of holding the particulate-containing fluid F.

FIG. 6 shows a filter washing assembly 100 according to another embodiment of the present invention. The filter washing assembly 100 is similar to the previous embodiment except the filter 140 of the filter washing assembly 100 includes a roll of material 142 contained in a canister 144. The roll of material 142 may be any material suitable for capturing biological particles such as, for example, polyester felt, a porous membrane material, or a glass fiber material. To enable the roll of material 142 to be inserted into the housing 120, the housing 120 may include, for example, a slot 124 that extends the entire width W of the housing 120 and communicates with a chamber 130 in the housing 120. When the housing 120 is closed, the roll of material 142 may be inserted into the slot 124 and advanced in a direction D until a portion of the roll of material is received in the chamber 130.

In operation, the roll of material 142 may be continuously fed into the chamber 130 through the slot 124. For example, particulate may be captured on a portion of the roll of material 142 that is upstream from the filter washing assembly 100 or may be captured on the roll of material 142 prior to inserting the roll of material 142 into the slot 124. The roll of material 142 may then be advanced in the direction D until the portion containing the sample particulate is disposed in the chamber 130. The filter 140 may then be washed substantially as described above to generate a first liquid sample. The continuous nature of the roll of material 142 permits a second particulate sample to be collected on another upstream portion of the roll of material 142. The roll of material 142 may then be advanced through the chamber 130 so that the portion containing the second sample is received in the chamber 130. A second liquid sample may then be generated substantially as described above. The filter washing assembly 100 may also include a sealing mechanism to prevent fluid from leaking out of the slot 124 during the wash process. For example, the filter washing assembly 100 may include a stopper configured to be inserted into slot 124 to seal the slot 124. After the washing steps are completed, the housing 120 may be opened to drain fluid from the chamber 130.

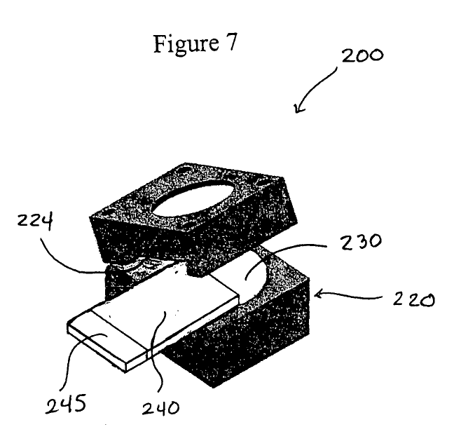

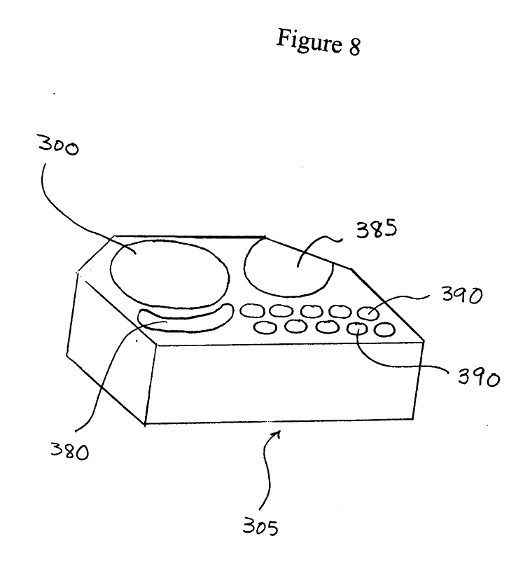

FIG. 7 shows a filter washing assembly 200 according to another embodiment of the present invention. The filter washing assembly 200 is similar to the previous embodiment except the filter 240 of the filter washing assembly 200 is disposed on a card 245 that is configured to be inserted into the housing 220 via a slot 224 that communicates with a chamber 230. The card 245 may be inserted into and removed from the slot 224 when the housing 220 is closed. Alternatively, the card 245 may be positioned in the housing 220 when the housing 220 is open. When the housing 220 is closed, the card 245 may be positioned in the slot 224 and clamped in place by pressure exerted on the card 245 by the two halves of the housing. The card 245 is removed from the housing 220 by opening the housing 220 slightly. As with the previous embodiments, the filter 240 may be any material suitable for capturing biological particles such as, for example, polyester felt, a porous membrane material, or a glass fiber material.

In operation, particulate may be captured on the filter 240. The card 245 may then be positioned in the slot 224 so that the filter 240 is received in the chamber 230. The filter 240 may be washed substantially as described above to generate a first liquid sample. After the card 245 is removed from the slot 224, another card 245 (or the same card 245 but containing a new filter 240) having a second particulate sample may then be positioned in the slot 224. A second liquid sample may then be generated substantially as described above. The filter washing assembly 200 may also include a sealing mechanism to prevent fluid from leaking out of the chamber 230 through the slot 224 during the wash process. For example, the filter washing assembly 200 may include a stopper configured to be inserted into a gap between the card 245 and the slot 224. Alternatively, the card 245 may be sized so that the slot 224 is substantially sealed when the card is positioned in the slot 224.

FIG. 8 shows another filter washing assembly 300 according to an embodiment of the present invention. The filter washing assembly 300 is similar to the previous embodiments except the filter washing assembly 300 is integrated into a cartridge 305. In addition to the filter washing assembly 300, the cartridge 305 may include, for example, a reservoir 380 for receiving the particulate laden fluid (i.e., the liquid sample) from the filter washing assembly 300. The cartridge 305 may also include at least one cavity (or mixing chamber) 385 configured to receive the liquid sample so that the liquid sample can be mixed with a reagent and/or a buffer. The cartridge 305 may include additional cavities 390 for holding various reagents and/or buffers as well as additional mixing chambers and chambers in which the liquid sample may undergo thermal cycling and analysis to identify the biological particulate washed from the filter. A filter washing assembly according to the present invention may also be adapted for use with existing filter washing systems and/or cartridges such as, for example, the fluid control and processing system disclosed in U.S. Pat. No. 6,374,684, incorporated by reference herein.

According to the above-described embodiments, a filter washing assembly is provided for generating a liquid sample. The filter washing assembly is configured to capture airborne biological particles (i.e., bioaerosols) on a filter and to generate the liquid sample by washing the filter to release the biological particles into a fluid.

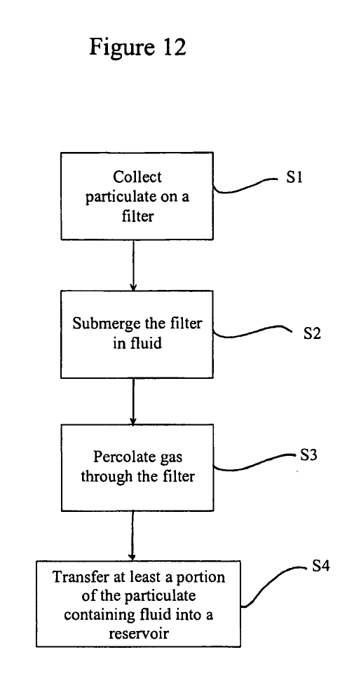

In operation, a method for generating a liquid sample according to an embodiment of the present invention includes the following steps, as shown in FIG. 12. The steps shown in FIG. 12 may be performed manually by an operator and/or may be automated. In step S1, a particulate 45 is collected on the filter 40 of the filter washing assembly 10. For example, the particulate 45 may be collected by passing a flow of air through the filter 40 in a first direction F1. In step S2, the filter 40 is submerged in a fluid F. In step S3, a gas (e.g., air) is percolated through the fluid and the filter 40. The gas is percolated in a direction F2 that is opposite the direction F1 so that the particulate 45 is washed (or dislodged) from the filter 40 into the fluid F above the filter 40. In this manner, the fluid above the filter 40 becomes laden with the particulate 45. In step S4, at least a portion of the particulate containing fluid is transferred into a reservoir 80 to thereby isolate the liquid sample. After the liquid sample is obtained, the liquid sample may be further processed and analyzed in any known manner. For example, the liquid sample may be purified to recover deoxyribonucleic acid (DNA) from the particulate 45, mixed with buffers and/or reagents, and analyzed in an identification module to identify the particulate to determine whether the particulate presents a biohazard. The identification module may include, for example, a lateral flow assay strip reader, a thermal cycler, a luminometer, and/or a surface plasmon resonance detector.

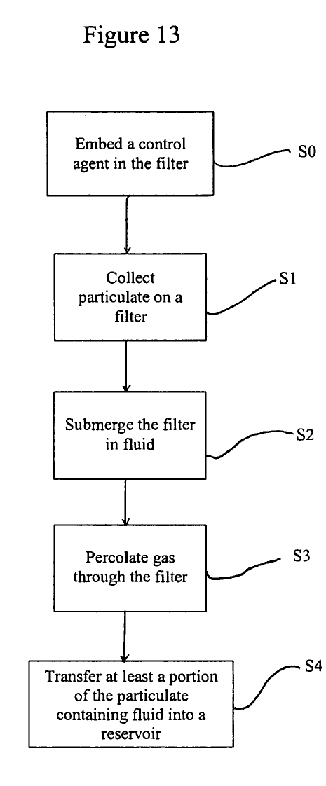

Another embodiment of a method for generating a liquid sample is shown in FIG. 13. The method of FIG. 13 is identical to the method of FIG. 12 except the method of FIG. 13 includes the use of a control agent 47 to verify proper washing of the filter. Specifically, FIG. 13 includes step SO prior to step S1. In step S0, a control agent is embedded in the filter. When gas is percolated through the filter in step S3, at least a portion of the particulate 45 and a portion of the control agent 47 are washed from the filter into the fluid F above the filter. In this manner, the fluid above the filter 40 becomes laden with the particulate 45 and the control agent 47.

Thus, according to the above embodiments, the present invention provides a filter washing assembly for capturing airborne particulate on a dry filter device and washing the filter to release the particulate from the filter to thereby generate a liquid sample. As a result, collection and analysis procedures may, for example, be automated and integrated into the collection system thereby reducing the logistical burden associated with manually collecting and analyzing the filters. The automated and integrated system may also be suitable for use in non-laboratory environments.

Additionally, the use of a dry filter device as opposed to a wet-walled aerosol collector or similar device has several advantages. For example, fluid evaporation during operation in a high temperature environment may be reduced because the fluid is exposed to the high temperature for a smaller amount of time. Accordingly, less fluid is required for a dry filter device. A dry filter device may also require less power for operation in low temperature environments because the dry filter device does not require the collection fluid to be heated during collection. Moreover, dry filter devices may have a much higher retention factor than wet-walled aerosol collectors or similar devices so that a greater sample volume is collected during a collection period.

Given the disclosure of the present invention, one versed in the art would appreciate that there may be other embodiments and modifications within the scope of the invention. Accordingly, all modifications attainable by one versed in the art from the present disclosure within the scope of the present invention are to be included as further embodiments of the present invention. The scope of the present invention is to be defined as set forth in the following claims.

1. A system for generating a liquid sample, comprising:
a chamber adapted to hold a fluid;
an air filter configured to be received in the chamber;
a mechanism for releasing at least a portion of a particulate disposed on the filter into the fluid located in the chamber; and
a structure for removing at least a portion of the particulate containing fluid from the chamber.

2. The system of claim 1, wherein the filter is configured to collect the particulate as a flow of air is passed through the filter.

3. The system of claim 1, wherein the filter includes an embedded control agent to verify proper operation of the system.

4. The system of claim 3, wherein the control agent comprises polystyrene beads with bound deoxyribonucleic acid segments and/or a fluorescent dye.

5. The cartridge of claim 1, wherein the filter includes a roll of material contained in a canister.

6. The cartridge of claim 1, wherein the filter is disposed on a card configured to be inserted into the chamber.

7. The cartridge of claim 1, wherein the filter is configured to be inserted in and removed from the chamber.

8. The system of claim 1, wherein the mechanism includes an air pump adapted to percolate air through the fluid and the filter.

9. The system of claim 8, wherein the filter is configured to collect the particulate as a flow of air is passed through the filter in a first direction and wherein the air pump is adapted to percolate the air through the filter in a second direction that is opposite to the first direction.

10. The system of claim 1, wherein the mechanism includes a sonicator.

11. The system of claim 1, wherein the mechanism includes an agitator adapted to mechanically agitate the filter.

12. The system of claim 1, wherein the structure includes an outlet communicating with the chamber and disposed adjacent to and substantially level with the filter so that particulate containing fluid disposed above the filter can flow from the chamber to the outlet.

13. The system of claim 1, further comprising a device for transferring at a portion of the particulate containing fluid from the chamber.

14. The system of claim 13, wherein the device includes an aspirator, a peristaltic pump, or a solenoid metering pump.

15. The system of claim 1, further comprising a module for analyzing the liquid sample.

16. The system of claim 15, wherein the module includes a lateral flow assay strip reader, a thermal cycler, a luminometer and/or a surface plasmon resonance detector.

17. The system of claim 1, further comprising a reservoir for collecting the particulate containing fluid from the chamber.

18. The system of claim 1, wherein a height of the system is approximately 2 inches, a width of the system is approximately 2.25 inches, and a length of the system is approximately 2.75 inches.

19. An cartridge for processing a liquid sample, comprising:
a chamber adapted to hold a fluid;
a filter received in the chamber;
an inlet for percolating air through the filter to thereby release a particulate disposed on the filter into the fluid; and
an outlet for transferring the particulate containing fluid from the chamber.

20. The cartridge of claim 19, further comprising a fan for moving air through the filter to capture the particulate on the filter.

21. The cartridge of claim 19, further comprising an air pump connected to the inlet.

22. The cartridge of claim 19, further comprising a reservoir connected to the outlet.

23. The cartridge of claim 19, further comprising at least one cavity configured to receive the liquid sample so that the liquid sample can be mixed with a reagent and/or a buffer

24. A method for generating a liquid sample, comprising:
collecting a particulate on a filter;
submerging the filter in a fluid;
percolating a gas through the filter so that the particulate is washed from the filter into the fluid; and
transferring at least a portion of the particulate containing fluid into a reservoir to thereby generate the liquid sample.

25. The method of claim 24, wherein at least one of the steps of collecting the particulate on the filter, submerging the filter in the fluid, percolating air through the filter, and transferring at least a portion of the particulate containing fluid is automated.

26. The method of claim 24, wherein the particulate is collected on the filter by passing a flow of air through the filter in a first direction.

27. The method of claim 26, wherein a direction of percolation of the gas is in a second direction that is opposite the first direction.

28. The method of claim 24, further comprising,
providing a control agent configured to verify proper washing of the filter; and
embedding the control agent in the filter so that at least a portion of the control agent is washed off the filter into the fluid when the gas is percolated through the filter.

29. The method of claim 24, further comprising purifying the liquid sample to recover deoxyribonucleic acid from the particulate.

30. The method of claim 24, further comprising mixing the liquid sample with buffers and/or reagents.

31. The method of claim 24, further comprising analyzing the liquid sample to thereby identify the particulate.
